(19) United States
(12) Patent Application Publication
(10) Pub. No.: US 2026/0117210 A1
(43) Pub. Date: Apr. 30, 2026

(54) RECOMBINANT CUTINASE EXPRESSION

(71) Applicant: Novozymes A/S, Bagsvaerd (DK)

(72) Inventors: Michael Dolberg Rasmussen, Vallensbaek (DK); Che Lin Kim, Copenhagen (DK); Christina Spuur Noedvig, Smoerum (DK)

(73) Assignee: Novozymes A/S, Bagsvaerd (DK)

(21) Appl. No.: 18/571,661

(22) PCT Filed: Jul. 11, 2022
(86) PCT No.: PCT/EP2022/069252

§ 371 Date: Dec. 18, 2023

(30) Foreign Application Priority Data
Jul. 13, 2021 (DK) PA202100758

(51) Int. Cl. C12N 9/18; C07K 14/32

(52) U.S. Cl. CPC C12N 9/18; C07K 14/32; C07K 2319/02; C12Y 301/01074

(57) ABSTRACT

The present invention relates to nucleic acid constructs comprising a first polynucleotide encoding a signal peptide from a bacterial DUF3298 domain-containing polypeptide and a second polynucleotide encoding a polypeptide having cutinase activity; expression vectors and host cells comprising said nucleic acid constructs; and methods for producing polypeptides having cutinase activity.

### REFERENCE TO A SEQUENCE LISTING

This application contains a Sequence Listing in computer-readable form, which is incorporated herein by reference.

### FIELD OF THE INVENTION

The present invention relates to nucleic acid constructs comprising a first polynucleotide encoding a signal peptide from a bacterial DUF3298 domain-containing polypeptide and a second polynucleotide encoding a polypeptide having cutinase activity; expression vectors and host cells comprising said nucleic acid constructs; and methods for producing polypeptides having cutinase activity.

### BACKGROUND OF THE INVENTION

Product development in industrial biotechnology includes a continuous challenge to increase enzyme yields at large scale to reduce costs. Two major approaches have been used for this purpose in the last decades. The first one is based on classical mutagenesis and screening. Here, the specific genetic modification is not predefined, and the main requirement is a screening assay that is sensitive to detect increments in yield. High-throughput screening enables large numbers of mutants to be screened in search for the desired phenotype, i.e., higher enzyme yields. The second approach includes numerous strategies ranging from the use of stronger promoters and multi-copy strains to ensure high expression of the gene of interest to the use of codon-optimized gene sequences to aid translation. However, high-level production of a given protein may in turn trigger several bottlenecks in the cellular machinery for secretion of the enzyme of interest into the medium, emphasizing the need for further optimization strategies.

Signal peptides (SPs) are short amino acid sequences present in the amino terminus of many newly synthesized polypeptides that target these into or across cellular membranes, thereby aiding maturation and secretion. The amino acid sequence of the SP influences secretion efficiency and thereby the yield of the polypeptide manufacturing process. Bioinformatic tools such as SignalP and SignalP5 can predict SPs from amino acid sequences, but most cannot distinguish between various types of SPs (Armenteros et al., Nat. Biotechnol. 37:420-423, 2019). Moreover, a large degree of redundancy in the amino acid sequence of SPs makes it difficult to predict the efficiency of any given SP for production of enzymes at industrial scale. Hence, SP selection is an important step for manufacturing of recombinant proteins, but the optimal combination of signal peptide and mature protein is very context dependent and not easy to predict.

Cutinases are lipolytic/esterolytic enzymes that catalyze the hydrolysis of insoluble triglycerides and a variety of polymers including cutin, an insoluble polymeric compound of plant cuticle. It has recently received extensive attention due to its capability to hydrolyze of polyethylene terephthalate (PET), which provides an enzymatic route for sustainable recycling of PET in packaging and textiles. Among various cutinases, leaf and branch compost cutinase (LCC) may be the most promising enzyme with high PET catalytic activity and thermostability. Engineered LCCs for higher activity and thermostability, and their application in the recycling of PET bottles, are described by Tournier et al. (Nature 580, 216-219 (2020) and in WO2015173265.

Although recombinant cutinase expression has been reported previously (Ferreira et al., Appl Microbiol Biotechnol (2003) 61:69-76), albeit with relatively low yields, in order to satisfy the growing demand within sustainable recycling of PET and other industries, it is necessary to provide recombinant expression systems with increased cutinase yields.

### SUMMARY OF THE INVENTION

The present invention is based on the surprising and inventive finding that expression of several cutinases with a signal peptide (SP2 32) obtained from a bacterial DUF3298 domain-containing polypeptide provides an improved yield of the cutinases compared to expression of the same cutinases with other signal peptides, i.e. a 2.2-fold to 4.6-fold increase in cutinase expression. Notably, the increased cutinase yields were achieved using several, different fermentation protocols and were observed for clones having either one or two copies of the expression cassettes integrated in their genome. The SP32 signal peptide was identified as one of several promising signal peptide sequences for cutinase expression. However, amongst the tested signal peptides, SP32 was the only signal peptide which showed highly increased cutinase yields after fermentation scale-up, which was totally unexpected.

In a first aspect, the present invention relates to nucleic acid constructs comprising:
a) first polynucleotide encoding a signal peptide, wherein the signal peptide has a sequence identity of at least 60% to SEQ ID NO:2; and
b) a second polynucleotide encoding a polypeptide having cutinase activity;
wherein the first polynucleotide and the second polynucleotide are operably linked in translational fusion.

In a second aspect, the present invention relates to expression vectors comprising nucleic acid constructs of the first aspect.

In a third aspect, the present invention relates to bacterial host cells comprising nucleic acid constructs of the first aspect and/or expression vectors of the second aspect.

In a fourth aspect, the present invention relates to methods for producing polypeptides having cutinase activity.

In a fifth aspect, the present invention relates to the use of a fermentation broth in a PET-degradation process, wherein the broth comprises a polypeptide having cutinase activity and a host cell according to the third aspect.

### BRIEF DESCRIPTION OF THE DRAWINGS

FIG. 1 shows a plasmid map of pCLK015

FIG. 2 shows a SDS-PAGE of cutinase expression (lane 1 and lane 7: protein ladders; lanes 2 and 3: X1 cutinase standard; lane 4: AN2781; lane 5: BT18062; lane 6: BT18062)

FIG. 3 shows a plasmid map of pAN2768

FIG. 4 shows a plasmid map of pAN2770

FIG. 5 shows a plasmid map of pBT18089

FIG. 6 shows a plasmid map of pBT18090

FIG. 7 shows screening of B. licheniformis SP library.

FIG. 8 shows relative cutinase expression for different SP sequences.

### SEQUENCE OVERVIEW

SEQ ID NO:1 is the SP32 signal peptide coding sequence.
SEQ ID NO:2 is SP32 signal peptide.
SEQ ID NO:3 is the DUF3298 domain-containing polypeptide coding sequence from B. pumilus.
SEQ ID NO:4 is DUF3298 domain-containing polypeptide from B. pumilus.
SEQ ID NO:5 is the cutinase X1 coding sequence.
SEQ ID NO:6 is cutinase X1.
SEQ ID NO:7 is the amyL signal peptide (SPamyL) coding sequence.
SEQ ID NO:8 is the amyL signal peptide (SPamyL).
SEQ ID NO:9 is the SP32-X1 coding sequence (with additional C-terminal Ala of SP32)
SEQ ID NO:10 is SP32-X1 (with additional C-terminal Ala of SP32)
SEQ ID NO:11 is the SPamyL-X1 coding sequence (with additional C-terminal Ala of SPamyL)
SEQ ID NO:12 is SPamyL-X1 (with additional C-terminal Ala of SPamyL)
SEQ ID NO:13 is SP32 signal peptide coding sequence. (with additional C-terminal Ala)
SEQ ID NO:14 is SP32 signal peptide. (with additional C-terminal Ala)
SEQ ID NO:15 is cryIIIA mRNA stabilizer region
SEQ ID NO: 16 is the SP32-X1 expression cassette
SEQ ID NO: 17 is the SPamyL-X1 expression cassette

### Definitions

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Cutinase: The term “cutinase” and the abbreviations “X1”, “X2” or “X3” means a polypeptide having cutinase activity (EC 3.1.1.74), such as polyethylene terephthalate (PET) hydrolase activity, that catalyzes the hydrolysis of cutin and/or the hydrolysis of p-nitrophenyl esters of hexadecenoic acid. For purposes of the present invention, cutinase activity, i.e. PET hydrolase activity, may be determined according to the procedures described in the “Materials and Methods” section of the Examples. The terms “cutinase”, “X1”, “X2”, “X3”, “cutinase variant” and “polypeptide having cutinase activity” are used interchangeably herein.

DUF3298 domain: The term “DUF3298 domain” or “DUF3298 domain-containing polypeptide” means a polypeptide comprising a domain of unknown function (DUF) 3298. DUF3298 represents a highly conserved domain found in a group of bacterial proteins. This group of bacterial protein C-terminal regions is highly conserved, but the function is not known. Several members are predicted as being endo-1,4-beta-xylanase-like. Proteins containing this domain include pdaC (EC 3.5.1.-), and anti-sigma-V factor RsiV from Bacillus subtilis as well as the DUF3298 domain-containing polypeptide of SEQ ID NO:4 from Bacillus pumilus. For the purpose of this invention, the signal peptide sequence of the DUF3298 domain-containing polypeptide of SEQ ID NO:4 is named “SP32” comprising or consisting of the amino acid sequence shown in SEQ ID NO:2.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Extension: The term “extension” means an addition of one or more amino acids to the amino and/or carboxyl terminus of a polypeptide, wherein the “extended” polypeptide has cutinase activity. Persons skilled in the art will know that a polypeptide having a given amino acid sequence and enzymatic activity may be produced with one or a few additional amino acids at the N- and/or C-terminus, and that such a polypeptide can have essentially the same enzyme activity. Such extended polypeptides are intended to be encompassed by the present invention.

Fragment: The term “fragment” as used in the context of a polypeptide means a polypeptide having one or more amino acids absent from its amino and/or carboxyl terminus, wherein the fragment has cutinase activity. The fragment may be produced naturally during expression and/or purification of the polypeptide, or may be the result of expression of a modified nucleotide sequence expressing the fragment or of targeted removal of amino acids from the amino and/or carboxy terminus.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., a promoter or domain of a polypeptide or nucleic acid, is not naturally associated with the polypeptide or nucleic acid, i.e., the control sequence is from a gene other than the gene encoding the mature polypeptide.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture or broth containing the secreted polypeptide.

Mature polypeptide: The term “mature polypeptide” means a polypeptide in its mature form following translation and any post-translational modifications such as N-terminal processing (e.g. removal of signal peptide), C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g. having a different C-terminal and/or N-terminal amino acid) as compared to another host cell expressing the same polynucleotide. Mature polypeptides of the invention may therefore have slight differences at the N- and/or C-terminal due to such differentiated expression by the host cell. A mature polypeptide having one or more amino acids absent from the N- and/or C-terminal may be considered to be a “fragment” of the full-length polypeptide. In some aspects, the mature polypeptide is amino acids 1 to 258 of SEQ ID NO:6 and amino acids −28 to −1 of SEQ ID NO:6 are a signal peptide.

Mature polypeptide coding sequence: The term “mature polypeptide coding sequence” means a polynucleotide that encodes a mature polypeptide having cutinase activity. In some aspects, the mature polypeptide coding sequence is nucleotides 88 to 864 of SEQ ID NO:9 and nucleotides 1 to 84 of SEQ ID NO:9 encode a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Obtained polypeptide/peptide/polynucleotide: The term “obtained” or “derived” when used in reference to a polynucleotide sequence, polypeptide sequence, cutinase sequence, variant sequence or signal peptide sequence, means that the molecule originally has been isolated from the given source and that the molecule can either be utilized in its native sequence or that the molecule is modified by methods known to the skilled person.

Operably linked: The term “operably linked” means a configuration in which a control sequence is placed at an appropriate position relative to the coding sequence of a polynucleotide such that the control sequence directs expression of the coding sequence.

Parent: The term “parent” means a polypeptide functioning as a signal peptide, or a polypeptide having cutinase activity, to which an alteration is made to produce variants of the present invention. The parent may be a naturally occurring (wild-type) polypeptide or a variant or fragment thereof.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g. a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the-nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows: (Identical Deoxyribonucleotides x 100)/(Length of Alignment-Total Number of Gaps in Alignment) (Identical Deoxyribonucleotides×100)/(Length of the Alignment)

Variant: The term “variant” means a polypeptide functioning as a signal peptide, or a polypeptide having cutinase activity, comprising a substitution, an insertion (including extension), and/or a deletion (including truncation), at one or more positions compared to the parent. A substitution means replacement of the amino acid occupying a position with a different amino acid; a deletion means removal of the amino acid occupying a position; and an insertion means adding one or more amino acids, e.g. 1-5 amino acids, adjacent to and immediately following the amino acid occupying a position.

Wild-type: The term “wild-type” in reference to an amino acid sequence or nucleic acid sequence means that the amino acid sequence or nucleic acid sequence is a native or naturally occurring sequence. As used herein, the term “naturally occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in a laboratory or by modification of the wild-type sequence).

### DETAILED DESCRIPTION OF THE INVENTION

The present invention is based on the surprising and inventive finding that expression of cutinases with a signal peptide from a bacterial DUF3298 domain-containing polypeptide provides an improved yield of the cutinases compared to expression of the same cutinases with other signal peptides.

As can be seen from the Examples disclosed herein, use of the signal peptide “SP32” (SEQ ID NO:2) from a bacterial DUF3298 domain-containing polypeptide (SEQ ID NO:4) provides an improved yield of several cutinases. Based on this observation, the present inventors expect a similar improvement for other cutinases and/or other signal peptides obtained or derived from bacterial DUF3298 domain-containing polypeptides.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

In a first aspect, the present invention relates to a nucleic acid construct comprising:
a) a first polynucleotide encoding a signal peptide, wherein the signal peptide has a sequence identity
of at least 60% to SEQ ID NO:2; and
b) a second polynucleotide encoding a polypeptide having cutinase activity;
   wherein the first polynucleotide and the second polynucleotide are operably linked in translational fusion.

The second polynucleotide is located downstream from the first polynucleotide. In one embodiment, the signal peptide is a naturally occurring signal peptide, or a functional fragment or functional variant of a naturally occurring signal peptide.

The signal peptide may be from any bacterial DUF3298 domain-containing polypeptide. In one embodiment, the signal peptide is from a DUF3298 domain-containing polypeptide expressed by a Bacillus species; preferably the signal peptide is derived from a DUF3298 domain-containing polypeptide expressed by a Bacillus species selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells; more preferably the signal peptide is derived from a DUF3298 domain-containing polypeptide expressed by Bacillus licheniformis, Bacillus subtilis or Bacillus pumilus; most preferably the signal peptide is from a DUF3298 domain-containing polypeptide expressed by Bacillus pumilus.

In one embodiment the signal peptide is derived from a bacterial DUF3298 domain-containing polypeptide having a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO:4; preferably the bacterial DUF3298 domain-containing polypeptide comprises, consists essentially of, or consists of SEQ ID NO:4. More preferably the signal peptide has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO:2. Most preferably the signal peptide comprises, consists essentially of, or consists of SEQ ID NO:2.

In some embodiments, the signal peptide is the signal peptide of the DUF3298 domain-containing polypeptide having an additional Ala at the C-terminus compared to SEQ ID NO:2, such as the signal peptide of SEQ ID NO: 14. In like manner, in some embodiments, the first polynucleotide encoding the signal peptide has an additional GCG codon at the 3′ end of the signal peptide coding region compared to SEQ ID NO:1, such as the signal peptide coding sequence of SEQ ID NO:13.

It is expected that the invention will be just as effective when employing a signal peptide that is highly similar to the signal peptide disclosed in SEQ ID NO:2 and encoded by SEQ ID NO: 1. One or more non-essential amino acids may, for example, be altered. Non-essential amino acids in a signal peptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for signal peptide activity to identify amino acid residues that are critical to the activity of the molecule and residues that are non-essential. See also, Hilton et al., 1996, J. Biol. Chem. 271:4699-4708. The identity of essential and non-essential amino acids can also be inferred from an alignment with one or more related signal peptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g. Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

Thus, in a preferred embodiment, the signal peptide has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO:2; most preferably the signal peptide comprises, consists essentially of, or consists of SEQ ID NO:2.

In a preferred embodiment, the polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 1; most preferably the polynucleotide comprises, consists essentially of, or consists of SEQ ID NO: 1.

In one aspect, the signal peptide is a variant (i.e., functional variant) or fragment (i.e., functional fragment) of the signal peptide of SEQ ID NO:2. In one aspect, the number of alterations in the signal peptide variant of the present invention is 1-10, e.g., 1-5, such as 1, 2, 3, 4, or 5 alterations. Alterations includes substitutions, insertions, and/or deletions at one or more (e.g., several) positions compared to the parent. A substitution means replacement of the amino acid occupying a position with a different amino acid; a deletion means removal of the amino acid occupying a position; and an insertion means adding an amino acid adjacent to and immediately following the amino acid occupying a position.

In a preferred embodiment, the signal peptide is a variant of the mature polypeptide of SEQ ID NO: 2 comprising 1-10 alterations, e.g., 1-5, such as 1, 2, 3, 4, or 5 alterations, compared to SEQ ID NO: 2.

The polypeptide having cutinase activity may be any such polypeptide or fragment or variant thereof. In one embodiment, the polypeptide having cutinase activity is a microbial polypeptide; preferably a bacterial polypeptide. Other, non-limiting examples of available cutinases are disclosed in Sulaiman et al. (Appl Environ Microbiol. 2012), Egmond and Vlieg (Biochimie 2000, Vol 82:11, 1015-1021) and in EP2922906, or any functional variants thereof.

Similar and as described above in relation to the signal peptide, it is expected that the invention will be just as effective when employing a polypeptide having cutinase activity that is highly similar to the mature polypeptide of SEQ ID NO:6 (encoded by SEQ ID NO:5).

In one embodiment, the polypeptide having cutinase activity is obtained from Thermobifida cellulosilytica DSM44535, Thermobifida halotolerans, Thermobifida fusca, Thermobifida alba, Fusarium solani, Fusarium solani pisi, Bacillus subtilis, Humicola insolens, Glomerella cingulate, or Thielavia terrestris.

Thus, in a preferred embodiment, the polypeptide having cutinase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide of SEQ ID NO:6, most preferably the polypeptide having cutinase activity comprises, consists essentially of, or consists of the mature polypeptide of SEQ ID NO:6.

In one aspect, the polypeptide having cutinase activity is a variant (i.e., functional variant) or fragment (i.e., functional fragment) of the mature polypeptide of SEQ ID NO:6. In one aspect, the number of alterations in the variants of the present invention is 1-20, e.g. 1-10 and 1-5, such as 1, 2, 3, 4, 5, 6, 7, 8, 9 or 10 alterations. Alterations includes substitutions, insertions, and/or deletions at one or more (e.g. several) positions compared to the parent. A substitution means replacement of the amino acid occupying a position with a different amino acid; a deletion means removal of the amino acid occupying a position; and an insertion means adding an amino acid adjacent to and immediately following the amino acid occupying a position.

In a preferred embodiment, the polypeptide having cutinase activity is a variant of the mature polypeptide of SEQ ID NO:6 comprising 1-20 alterations, e.g. 1-10 and 1-5, such as 1, 2, 3, 4, 5, 6, 7, 8, 9 or 10 alterations, compared to SEQ ID NO:6.

Most preferably, the polypeptide having cutinase activity comprises or consists of the mature polypeptide of SEQ ID NO:6. In one embodiment, the polypeptide having cutinase activity comprises or consists of the mature polypeptide of SEQ ID NO:6 with an additional N-terminal Ala. Due to the degeneracy of the genetic code, different polynucleotides can encode the same polypeptide. Thus, in a preferred embodiment, the polynucleotide encoding the polypeptide having cutinase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:5; most preferably the polynucleotide comprises, consists essentially of, or consists of the mature polypeptide coding sequence of SEQ ID NO:5.

The first and second polynucleotide are operably linked in translational fusion. In the context of the present invention, the term “operably linked in translation fusion” means that the signal peptide encoded by the first polynucleotide and the polypeptide having cutinase activity encoded by the second polynucleotide are encoded in frame and translated together as a single polypeptide. Preferably, following translation, the signal peptide is removed to provide the mature polypeptide having cutinase activity. Alternatively, the signal peptide is not removed, or only removed partly to provide the mature polypeptide having cutinase activity and comprising at least a fragment of the signal peptide.

The first and second polynucleotide may be manipulated in a variety of ways to provide for expression of a variant. Manipulation of the polynucleotide prior to its insertion into a nucleic acid construct or expression vector may be desirable or necessary depending on the construct or vector. The techniques for modifying polynucleotides utilizing recombinant DNA methods are well known in the art.

Besides a signal peptide, the nucleic acid constructs of the invention may be operably linked to one or more further control sequences that direct the expression of the coding sequence in a suitable host cell under conditions compatible with the control sequences.

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in “Molecular Cloning: A laboratory manual” (2001, J. Sambrook and D. V. Russel) and by Y. Song et al. (2016) PLoS ONE 11 (7): e0158447.

In one embodiment, the nucleic acid construct further comprises a heterologous promoter, and wherein said promoter, the first polynucleotide, and the second polynucleotide are operably linked. The promoter is orientated upstream of the first polynucleotide.

In an embodiment, the promoter is a heterologous promoter. Preferably, the promoter is a tandem promoter. More preferably, the promoter is a P3 promoter or a P3-based promoter.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177:3465-3471).

In one embodiment, the promoter is a promoter, such as a P3 promoter, operably linked to an mRNA stabilizer region. Preferably, the mRNA stabilizer region is the cryIIIA mRNA stabilizer region, preferably the cryIIIA mRNA stabilizer region of SEQ ID NO: 15.

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rmnB).

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE) or Bacillus subtilis neutral protease (nprT).

Where both signal peptide and propeptide sequences are present, the propeptide sequence is positioned next to the N-terminus of a polypeptide and the signal peptide sequence is positioned next to the N-terminus of the propeptide sequence. Additionally, or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in bacterial systems include the lac, tac, and trp operator systems. Other examples of regulatory sequences are those that allow for gene amplification. In these cases, the polynucleotide encoding the polypeptide would be operably linked to the regulatory sequence.

The control sequence may also be a leader, a non-translated region of an mRNA that is important for translation by the host cell. The leader is operably linked to the 5′-terminus of the polynucleotide encoding the polypeptide. Any leader that is functional in the host cell may be used. Suitable leaders for bacterial host cells are described by Hambraeus et al., Microbiology 2000; 146 12:3051-3059, and by Kaberdin and Blasi, FEMS Microbiol Rev 2006, 30 (6): 967-79.

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e. by activating the transcription of the gene encoding the protein of interest by binding to its promoter, or indirectly, i.e. by activating the transcription of a further transcription factor which regulates the transcription of the gene encoding the protein of interest, such as by binding to the promoter of the further transcription factor. Suitable transcription factors for prokaryotic host cells are described in Seshasayee et al., Subcell Biochem 2011; 52:7-23, as well in Balleza et al., FEMS Microbiol Rev 2009, 33 (1): 133-151.

### Expression Vectors

In a second aspect, the present invention also relates to recombinant expression vectors comprising a nucleic acid construct according to the first aspect. The expression vectors comprise a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of the first and second polynucleotide of the present invention may be inserted into a host cell to increase production of a variant. In one embodiment, at least two copies are inserted into the genome of the host cell. An increase in the copy number of the first and second polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g. Sambrook et al., 1989, supra).

### Host Cells

In a third aspect, the invention relates to bacterial host cells comprising in its genome:
a) a nucleic acid construct according to the first aspect; and/or
b) an expression vector according to the second aspect.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source. The polypeptide encoded by the introduced polynucleotide can be native or heterologous to the recombinant host cell. Also, at least one of the one or more control sequences can be heterologous to the polynucleotide encoding the polypeptide. The recombinant host cell may comprise a single copy, or at least two copies, e.g. three, four, five or more copies of the polynucleotide of the present invention.

In one embodiment, the host cell comprises one copy of the nucleic acid construct and/or the expression vector.

In one embodiment, the host cell comprises two or more copies of the nucleic acid construct and/or the expression vector.

The host cell may be any bacterial cell useful in the recombinant production of a polypeptide of the present invention, e.g., Gram-positive or a Gram-negative bacterium.

In a preferred embodiment the host cell is a Gram-positive host cell.

Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

In one embodiment the host cell is a Bacillus cell; preferably a Bacillus cell selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cell; most preferably a Bacillus licheniformis cell.

Since the classification of Bacillus cells may change in the future, for the purposes of this invention, Bacillus classes/genera/species shall be defined as described in Patel and Gupta, Int. J. Syst. Evol. Microbiol. 2020; 70:406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending e.g. on the genus. Methods for introducing DNA into prokaryotic host cells are for example described in Heinze et al., 2018, BMC Microbiology 18:56, Burke et al., 2001, Proc. Natl. Acad. Sci. USA 98:6289-6294, Choi et al., 2006, J. Microbiol. Methods 64:391-397, and Donald, Guedon and Renault, 2013, Journal of Bacteriology, 195:11 (2612-2620).

In one embodiment, the bacterial host cell has increased cutinase yield relative to a control host cell lacking the signal peptide, being otherwise isogenic, when cultivated under identical conditions.

In one embodiment, the control host cell encodes a amyL signal peptide fused to the cutinase.

In one embodiment, the amyL signal peptide fused to the cutinase has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO:8; most preferably the amyL signal peptide comprises, consists essentially of, or consists of SEQ ID NO:8.

In one embodiment, the increased cutinase yield of the host cell is at least 1.5-fold, 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, at least 5-fold, least 5.1-fold, at least 5.2-fold, at least 5.3-fold, at least 5.4-fold, or at least 5.5-fold relative to the control host cell.

In one embodiment, the cutinase yield is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, at least 400%, at least 405%, at least 410%, at least 415%, at least 420%, at least 425%, at least 430%, at least 435%, at least 440%, at least 445%, at least 450%, at least 455%, at least 460%, at least 465%, at least 470%, at least 475%, at least 480%, at least 485%, at least 490%, at least 495%, or at least 500%, relative to the cutinase yield in the control host cell which does not comprise the SP32 signal peptide, when cultivated under identical conditions. Preferably, the control host cell is otherwise isogenic to the host cell.

In one embodiment, the increased cutinase yield is measured or achieved after a cultivation time of at least 24 hours, at least 48 hours, at least 72 hours, at least 96 hours, or at least 120 hours.

In one embodiment, the increased cutinase yield is measured or achieved after cultivation in batch-mode, fed-batch mode, or continuous mode, preferably fed-batch mode.

### Methods of Production

In a fourth aspect, the present invention also relates to methods of producing a polypeptide having cutinase activity, the method comprising:
a) cultivating a bacterial host cell according to the third aspect under conditions conducive for production of the polypeptide having cutinase activity; and optionally
b) recovering the polypeptide having cutinase activity.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, solid-state, and/or microcarrier-based fermentations) in laboratory or industrial fermentors in a suitable medium and under conditions allowing the polypeptide to be expressed and/or isolated, e.g. as described in below “Examples” or as described in WO2020/229191 (“Standard fed-batch cultivation procedure”, pages 69-70). Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

In one embodiment, the cultivation is a fed-batch process.

In one embodiment, the cultivation is carried out over a period of at least 48 hours, at least 72 hours, at least 96 hours, or at least 120 hours.

The polypeptide may be detected using methods known in the art that are specific for the polypeptides, including, but not limited to, the use of specific antibodies, formation of an enzyme product, disappearance of an enzyme substrate, or an assay determining the relative or specific polypeptide activity.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered.

The polypeptide and/or polypeptide fragments may be purified by a variety of procedures known in the art to obtain substantially pure polypeptides and/or polypeptide fragments (see, e.g., Wingfield P. T., 2015, Current Protocols in Protein Science; 80 (1) 6.1.1-6.1.35; Labrou N. E.; 2014, Protein Downstream Processing, 1129:3-10).

In an alternative aspect, the polypeptide having cutinase activity is not recovered, but rather a host cell of the present invention expressing the polypeptide having cutinase activity is used as a source of the variant.

### Fermentation Broth Formulations or Cell Compositions

The present invention also relates to a fermentation broth formulation or a cell composition comprising a polypeptide having cutinase activity. The fermentation broth product further comprises additional ingredients used in the fermentation process, such as, for example, cells (including, the host cells containing the nucleic acid constructs of the present invention which are used to produce the polypeptide having cutinase activity), cell debris, biomass, fermentation media and/or fermentation products. In some embodiments, the composition is a cell-killed whole broth containing organic acid(s), killed cells and/or cell debris, and culture medium.

In a fifth aspect, the invention relates to the uses of a fermentation broth in a PET degradation processes, the broth comprising a polypeptide having cutinase activity and a host cell according to the third aspect. In one embodiment, the fermentation broth is used directly in a PET degradation process, without further filtration or purification of the polypeptide having cutinase activity.

In an alternative aspect, the invention relates to a method of producing a whole broth formulation or cell culture composition comprising a polypeptide having cutinase activity, the method comprising:
a) cultivating a bacterial host cell according to the third aspect under conditions conducive for production of the polypeptide having cutinase activity; and
b) filtering the cultivation broth to recover the polypeptide having cutinase activity.

In one embodiment the filtered broth is used in a PET degradation process. The term “fermentation broth” as used herein refers to a preparation produced by cellular fermentation that undergoes no or minimal recovery and/or purification. In one embodiment, the fermentation broth is an ultra-filtrated fermentation broth. For example, fermentation broths are produced when microbial cultures are grown to saturation, incubated under carbon-limiting conditions to allow protein synthesis (e.g. expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g. filamentous fungal cells) are removed, e.g. by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g. bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells are grown to saturation, incubated under carbon-limiting conditions to allow protein synthesis. In some embodiments, the cell-killed whole broth or composition contains the spent cell culture medium, extracellular enzymes, and killed bacterial cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Examples

### Materials and Methods

Chemicals used as buffers and substrates were commercial products of at least reagent grade.

PCR amplifications were performed using standard textbook procedures, employing a commercial thermocycler and either Ready-To-Go PCR beads, Phusion polymerase, or RED-TAQ polymerase from commercial suppliers.
LB agar: See EP0506780, p.9.
LBPSG agar plates contain LB agar supplemented with phosphate (0.01 M K3PO4), glucose (0.4%), and starch (0.5%); See EP0805867, paragraph [0137].
TY: liquid broth medium; See WO94/14968, p. 16.
Cal18-2: liquid broth medium; see WO2000075344, p. 20-21
Oligonucleotide primers were obtained from Integrated DNA technologies, Leuven, Belgium. DNA manipulations (plasmid and genomic DNA preparation, restriction digestion, purification, ligation, DNA sequencing) was performed following the standard manufacturer's instructions with commercially available kits and reagents.

DNA was introduced into B. subtilis rendered naturally competent, either using a two-step procedure (Yasbin et al., 1975, J. Bacteriol. 121:296-304), or a one-step procedure, in which cell material from an agar plate was resuspended in Spizisen 1 medium (WO 2014/052630), 10 ml shaken at 200 rpm for approx. 4 hours at 37° C., DNA added to 400 microliter aliquots, and these further shaken 150 rpm for 1 hour at the desired temperature before plating on selective agar plates.

DNA was introduced into B. licheniformis by conjugation from B. subtilis, essentially as previously described (EP2029732), using a modified B. subtilis donor strain pp 3453, containing pLS20, wherein the methylase gene M.bli1904II (US20130177942) is expressed from a triple promoter at the amyE locus, the pBC16-derived orf beta and the B. subtilis comS gene (and a kanamycin resistance gene) are expressed from a triple promoter at the alr locus (making the strain D-alanine requiring), and the B. subtilis comK gene expressed from a mannose inducible promoter in the xylA locus.

All the constructions described in the examples were assembled from synthetic DNA fragments ordered from Integrated DNA technologies. The fragments were assembled by sequence overlap extension (SOE) as described in the examples. For plasmid construction was mainly used Prolonged Overlap Extension PCR (POE-PCR), which generates multimeric plasmids, as previously described (You et al. 2012. Simple cloning via direct transformation of PCR product (DNA multimer) to Escherichia coli and Bacillus subtilis. Appl. Environ. Microbiol. 78 (5): 1593-1595).

The temperature-sensitive plasmids used herein were incorporated into the genome of B. licheniformis by chromosomal integration and excision according to the method previously described (U.S. Pat. No. 5,843,720). B. licheniformis transformants containing plasmids were grown on LBPG selective medium with erythromycin at 50° C. to force integration of the vector at identical sequences to the chromosome. Desired integrants were chosen based on their ability to grow on LBPG+erythromycin selective medium at 50° C. Integrants were then grown without selection on LBPG plates at 34° C. to allow excision of the integrated plasmid. Cells were then grown in liquid LBPG medium at 37° C. for 6-8 hours. The cultures were then plated on LBPG plates and screened for erythromycin-sensitivity. The sensitive clones were checked for correct integration of the desired construct.

Genomic DNA was prepared from several erythromycin sensitive isolates above by using the commercially available QIAamp DNA Blood Kit from Qiagen.

### Standard Microplate Batch-Fermentation

The BioLector® is a microfermentation system that monitors online common fermentation parameters such as biomass, pH, oxygen saturation and fluorescence. It contains a temperature- and humidity-controlled incubation chamber that carries a single microplate. The fermentation can be monitored continuously by an optical fiber that moves below the plate. In this work, a BioLector® (m2p-Labs, Baesweiler, Germany) was used for the measurement of scattered light and GFP fluorescence. For Example 3 and Example 7, cultivations were performed in LB media, at a shaking frequency of 1000 rpm, 37° C. and 85% humidity in 48-well Flowerplates® (M2p-labs), covered with a Sealing Foil with Reduced Evaporation (M2p-Labs). Fermentations were carried out at least in biological triplicates for 72 hours, and the supernatants harvested for subsequent cutinase activity measurements.

### Cutinase Activity Assay

The cultivation samples from microplate fermentation or fed-batch fermentation were diluted. 20 μL of the diluted cultivation sample was transferred in technical duplicates to 96-well plates. A calibration curve with increasing concentrations of purified cutinase standard (SEQ ID NO:6, kindly provided by Carbios, France) was added to each 96 well plate. 180 ul of p-nitrophenyl palmitate (Sigma-Aldrich, Denmark) was added to the plate and the colorimetric reaction was measured in a Cytation5 plate reader at 405 nm, 23° C. for 5 min, measuring absorbance every 30 seconds.

### SDS-PAGE

At day three post-inoculation, cultures were centrifuged at 6000×g and the supernatants were collected. 20 μL of the culture supernatants were mixed with an NuPAGE® LDS sample buffer 4× (Invitrogen, Carlsbad, CA, USA) and heated to 70° C. for 10 minutes. The samples were analyzed on SDS-PAGE using a NuPAGER 4-12% Bis-Tris gel (Invitrogen) and Coomassie blue staining.

### Fed-Batch Cultivation Procedure

All growth media were sterilized by methods known in the art.

Inoculum steps: First the strain was grown on agar slants 1 day at 37° C. The agar was then washed with buffer, and the optical density (OD) at 650 nm of the resulting cell suspension was measured. The inoculum shake flask was inoculated with an inoculum of OD (650 nm)×ml cell suspension=0.1. The shake flask was incubated at 37° C. at 300 rpm for 20 hr. The fermentation in the main fermentor was started by inoculating the main fermentor with the growing culture from the shake flask. The inoculated volume was 11% of the medium (inorganic salts, protein hydrolysate, trace metals, and vitamins) (i.e. 80 ml for 720 ml media).

Standard lab fermentors were used equipped with a temperature control system, pH control with ammonia water and phosphoric acid, dissolved oxygen electrode to measure oxygen saturation through the entire fermentation. Feed medium: Sucrose 708 g/l.
Fermentation parameters: Temperature: 30-42° C.; The pH was controlled using ammonia water and phosphoric acid.
Aeration: 1.5 liter/min/kg broth weight.
Agitation: 1500 rpm.
Experimental setup: The cultivation was run for five days with constant agitation, and the oxygen tension was followed on-line in this period. The different strains were compared side by side.

### Strains

AEB1517: This strain is a B. subtilis donor strain for conjugation of B. licheniformis as described previously (see U.S. Pat. Nos. 5,695,976, 5,733,753, 5,843,720, 5,882,888, and WO2006042548). The strain contains pLS20 and the methylase gene M.blil 904II (US20130177942) expressed from a triple promoter at the amyE locus, the pBC16-derived orf beta and the B. subtilis comS gene (and a kanamycin resistance gene) are expressed from a triple promoter at the alr locus (making the strain D-alanine requiring).
pp 3724: AEB1517 derivative where a second gene cassette consisting of the comS gene expressed from a triple promoter is inserted at the pel locus (pectate lyase) (see US20190276855).
BT18049: pp 3724 transformed with the plasmid pCLK015.
AN2766: pp 3724 with plasmid pAN2766
AN2768: pp 3724 with plasmid pAN2768
AN2770: pp 3724 with plasmid pAN2770
BT18089: pp 3724 with plasmid pBT18089
BT18090: pp 3724 with plasmid pBT18090

AN1302: A derivative of B. licheniformis Ca63 with seven deletions in the protease genes, aprL, mprL, bprAB, epr, wprA, vpr, ispA. Furthermore, a deletion in the spo gene spollAC, the cypX, sacB and forD.

AN2781: AN1302 with two copies of the expression cassette encoding amyL signal peptide fused to the cutinase X1. The two copies are inserted at the lacA2 locus and the xylA locus on the chromosome.

BT18062: AN1302 with two copies of the expression cassette encoding SP32 signal peptide fused to the cutinase X1. The two copies are inserted at the lacA2 locus and the xylA locus on the chromosome.

AN2783: AN1302 with two copies of the expression cassette encoding amyL signal peptide fused to the cutinase X2. The two copies are inserted at the lacA2 locus and the xylA locus on the chromosome.

BT18014: AN1302 with two copies of the expression cassette encoding amyL signal peptide fused to the cutinase X3. The two copies are inserted at the lacA2 locus and the xylA locus on the chromosome.

BT18093: AN1302 with two copies of the expression cassette encoding SP32 signal peptide fused to the cutinase X2. The two copies are inserted at the lacA2 locus and the xylA locus on the chromosome.

BT18095: AN1302 with two copies of the expression cassette encoding SP32 signal peptide fused to the cutinase X3. The two copies are inserted at the lacA2 locus and the xylA locus on the chromosome.

MOL3320: Bacillus licheniformis, amyL, aprL, bgIC, cypX, forD, gntP, lacA2, mprL, sacB, spollAC, xylA, ara::Mad7d-sgRNA::mecA-ERM (as described in patent application US 2019/0185847 A1)

Bacillus licheniformis SP clones SP1 to SP85: MOL3320 with one copy of SP-cutinase fusion gene integrated in the ara locus. These clones were investigated in Examples 8-9.

### Plasmids

pAEB267: A pE194 derivative plasmid with a minimal attP site of TP901-1, previously described in US20080085535.

pAN2766: A pAEB267 derivative where the gene encoding signal peptide from alpha-amylase from B. licheniformis (SPamyL) is fused to the cutinase X1 gene.

pAN2768: A pAEB267 derivative where the gene encoding signal peptide from alpha-amylase from B. licheniformis (SPamyL) is fused to the cutinase X2 gene.

pAN2770: A pAEB267 derivative where the gene encoding signal peptide from alpha-amylase from B. licheniformis (SPamyL) is fused to the cutinase X3 gene.

pCLK015: pAEB267 derivative where the gene encoding the SP32 signal peptide is fused to the cutinase X1 gene.

pBT18089: A pAEB267 derivative where the gene encoding signal peptide SP32 is fused to the cutinase X2 gene.

pBT18090: A pAEB267 derivative where the gene encoding signal peptide SP32 is fused to the cutinase X3 gene.

### Example 1. Construction of Bacillus licheniformis Strain AN2781 Expressing Cutinase X1 with the amyL Signal Peptide

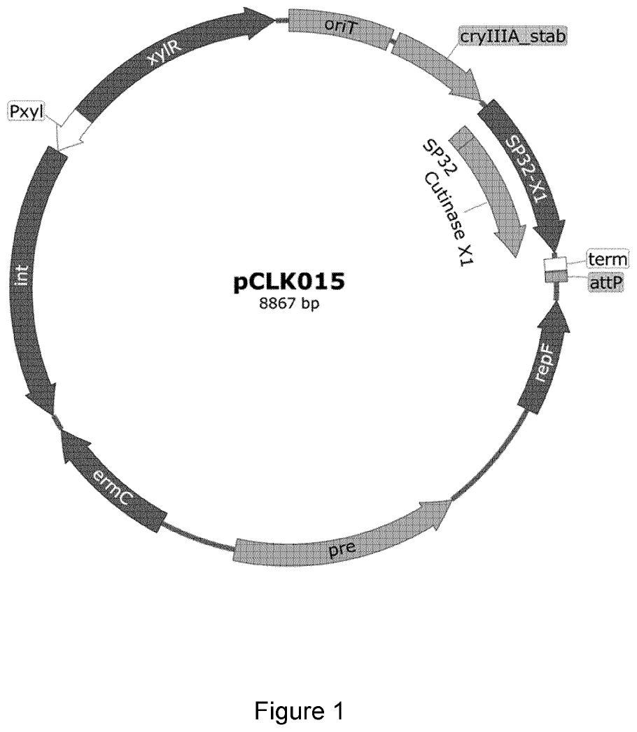

Plasmid pAN2766 was constructed for insertion of a gene encoding cutinase with the signal peptide of Bacillus licheniformis alpha-amylase (amyL; designated by gene name SPamyL-X1) into the genome of a Bacillus subtilis host using the site-specific recombinase-mediated method described in WO 2018/077796. A map of pAN2766 is shown in FIG. 1, the DNA sequence encoding cutinase X1 with the amyL signal peptide is shown in SEQ ID NO: 11 (comprising SEQ ID NO:5 encoding cutinase X1 and SEQ ID NO:7 encoding SPamyL), and the corresponding amino acid sequence is shown in SEQ ID NO: 12 (SPamyL-X1: comprising SPamyL with SEQ ID NO:8 and cutinase X1 with SEQ ID NO: 6). Cutinase X1 is previously described in WO2020/021118. Plasmid pAN2766 was introduced into conjugation donor strain Bacillus subtilis pp 3724 by transformation, resulting in strain AN2766 (U.S. Plant Pat. No. 3,724/pAN2766). Using conjugation donor strain AN2766, plasmid pAN2766 was introduced by conjugation into a derivative of Bacillus licheniformis AN1302 comprising two chromosomal target sites for insertion of the plasmid and deletions in the genes encoding alkaline protease (aprL), Glu-specific protease (mprL), bacillopeptidase F (bprAB), minor extracellular serine proteases (epr and vpr), secreted quality control protease (wprA) and intracellular serine protease (ispA). At each of the two chromosomal target sites of the B. licheniformis host is an expression cassette comprising a P3 promoter followed by the cryIIIA mRNA stabilizer region (SEQ ID NO: 15), a fluorescent marker gene and an attB recombination site. The corresponding SPamyL-X1 expression cassette is shown in SEQ ID NO: 17. The plasmid inserted into the B. licheniformis chromosome by site-specific recombination between the attP sites on the plasmid, and attB sites at the target chromosomal loci. The plasmid was then allowed to excise from the chromosome via homologous recombination by incubation at 34° C. in the absence of erythromycin selection. Integrants that had lost the plasmid were selected by screening for erythromycin sensitivity and loss of fluorescence marker phenotype. Integration of the SPamyL-X1 gene was confirmed by PCR analysis. One B. licheniformis integrant with the SPamyL-X1 gene inserted at two chromosomal loci was designated AN2781.

### Example 2. Construction of Bacillus licheniformis Strain BT18062 Expressing Cutinase X1 with SP32 Signal Peptide

Plasmid pCLK015 was constructed for insertion of a gene encoding cutinase with the signal peptide of Bacillus pumilus putative DUF3298 (designated by name SP32-X1) into the genome of a Bacillus subtilis host using the site-specific recombinase-mediated method described in WO 2018/077796. A map of pCLK015 is shown in FIG. 1, the DNA sequence encoding cutinase X1 with the SP32 signal peptide is shown in SEQ ID NO:9 (comprising SEQ ID NO:1 encoding the SP32 and SEQ ID NO:5 encoding the cutinase X1), and the corresponding amino acid sequence is shown in SEQ ID NO: 10 (SP32-X1: comprising SP32 with SEQ ID NO:2 and cutinase X1 with SEQ ID NO:6). Plasmid pCLK015 was introduced into conjugation donor strain Bacillus subtilis pp 3724 by transformation, resulting in strain BT18049 (U.S. Plant Pat. No. 3,724/pCLK015). Using conjugation donor strain BT18049, plasmid pCLK015 was introduced by conjugation into a derivative of Bacillus licheniformis AN1302 comprising two chromosomal target sites for insertion of the plasmid and deletions in the genes encoding alkaline protease (aprL), Glu-specific protease (mprL), bacillopeptidase F (bprAB), minor extracellular serine proteases (epr and vpr), secreted quality control protease (wprA) and intracellular serine protease (ispA). At each of the two chromosomal target sites of the B. licheniformis host is an expression cassette comprising a P3 promoter followed by the cryIIIA mRNA stabilizer region (SEQ ID NO:15), a fluorescent marker gene and an attB recombination site. The corresponding SP32-X1 expression cassette is shown in SEQ ID NO: 16. The plasmid inserted into the B. licheniformis chromosome by site-specific recombination between the attP sites on the plasmid, and attB sites at the target chromosomal loci. The plasmid was then allowed to excise from the chromosome via homologous recombination by incubation at 34° C. in the absence of erythromycin selection. Integrants that had lost the plasmid were selected by screening for erythromycin sensitivity and loss of fluorescence marker phenotype. Integration of the SP32-X1 gene was confirmed by PCR analysis. One B. licheniformis integrant with the SP32-X1 gene inserted at two chromosomal loci was designated BT18062.

### Example 3. Comparison of Cutinase Production by Bacillus licheniformis Integrants Expressing Cutinase X1 with Either the amyL Signal Peptide or with the SP32 Signal Peptide

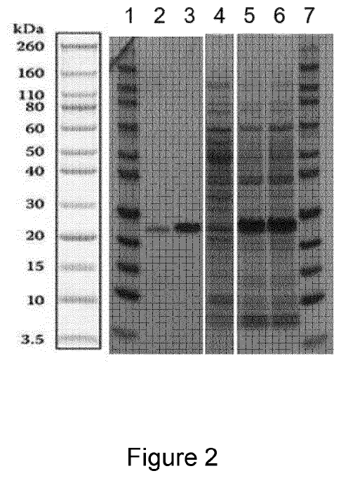

B. licheniformis strains AN2781 and BT18062 were tested with respect to cutinase productivity in batch cultivations in BioLector® as described above. Cutinase production by the strains was compared using enzyme activity assay and SDS-PAGE. Relative total cutinase product assessed by the activity assay are shown in Table 1, whereas the SDS-PAGE is shown in FIG. 2 (lane 1 and lane 7: protein ladders; lanes 2 and 3: X1 cutinase standard; lane 4: AN2781; lane 5: BT18062; lane 6: BT18062). As can be seen in Table 1, the amount of cutinase product was 2.3-fold increased in BT18062 with SP32 signal peptide relative to AN2781 with SPamyL signal peptide.

TABLE 1
Relative total cutinase product for B. licheniformis
strains expressing cutinase (N = 6)

| | Number of cutinase | Signal Peptide | Relative product |
|---|---|---|---|
| Strain | gene copies | Source | yield |
| AN2781 | 2 | amyL | 1.0 ± 0.2 |
| BT18062 | 2 | SP32 | 2.3 ± 0.2 |

### Example 4. Comparison of Cutinase Fed-Batch Production by Bacillus licheniformis Integrants Expressing Cutinase X1 with Either the Amyl Signal Peptide or with the SP32 Signal Peptide

B. licheniformis strains AN2781 and BT18062 were tested with respect to cutinase productivity in fed-batch cultivations as described above. Cutinase production by the strains was compared using enzyme activity assay. Relative total cutinase products are shown in Table 2. The amount of cutinase product was 4.6-fold increased in BT18062 with SP32 signal peptide relative to AN2781 with SPamyL signal peptide.

TABLE 2
Relative total cutinase product for B. licheniformis
strains expressing cutinase (N = 3)

| | Number of cutinase | Signal Peptide | Relative product |
|---|---|---|---|
| Strain | gene copies | Source | yield |
| AN2781 | 2 | amyL | 1.0 ± 0.3 |
| BT18062 | 2 | SP32 | 4.6 ± 0.4 |

### Example 5. Construction of Bacillus licheniformis Strains AN2783 and BT18014 Expressing Cutinase Variants with the amyL Signal Peptide

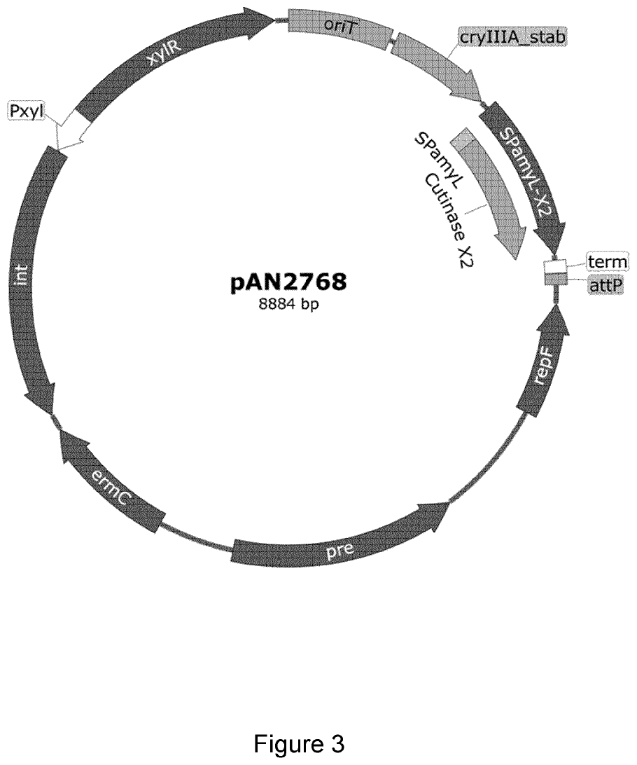

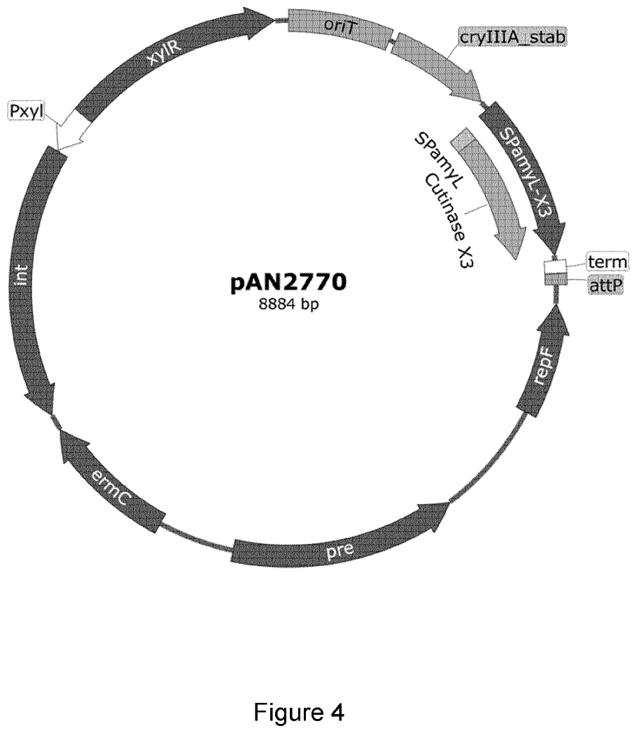

Plasmid pAN2768 and pAN2770 was constructed for insertion of a gene encoding cutinase variants X2 and X3 (both X2 and X3 are cutinase variants derived from cutinase X1) with the signal peptide of Bacillus licheniformis alpha-amylase (amyL; designated by gene name SPamyL-X2 and SPamyL-X3) into the genome of a Bacillus host using the site-specific recombinase-mediated method described above. Construction of expression cassettes for SPamyL-X2 and SPamyL-X3 has been carried out according to the construction of SPamyL-X1 described in Example 1. The maps of pAN2768 and pAN2770 are shown in FIG. 3 and FIG. 4, respectively. Plasmid pAN2768 and pAN2770 was introduced into conjugation donor strain Bacillus subtilis pp 3724 by transformation, resulting in strain AN2768 and AN2770. Using conjugation donor strain AN2768 or AN2770, plasmid pAN2768 or pAN2770 was introduced by conjugation into a derivative of Bacillus licheniformis AN1302 comprising two chromosomal target sites for insertion of the plasmid as described above. Integration of SPamyL-X2 or SPamyL-X3 gene was confirmed by PCR analysis. One B. licheniformis integrant with the SPamyL-X2 or SPamyL-X3 gene inserted at two chromosomal loci was designated AN2783 and BT18014, respectively.

### Example 6. Construction of Bacillus licheniformis Strains BT18093 and BT18095 Expressing Cutinase Variants with the SP32 Signal Peptide

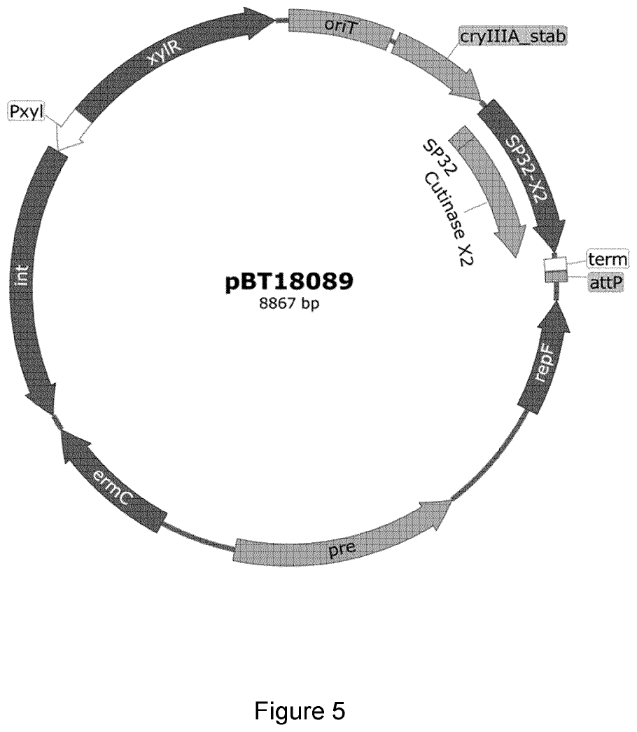

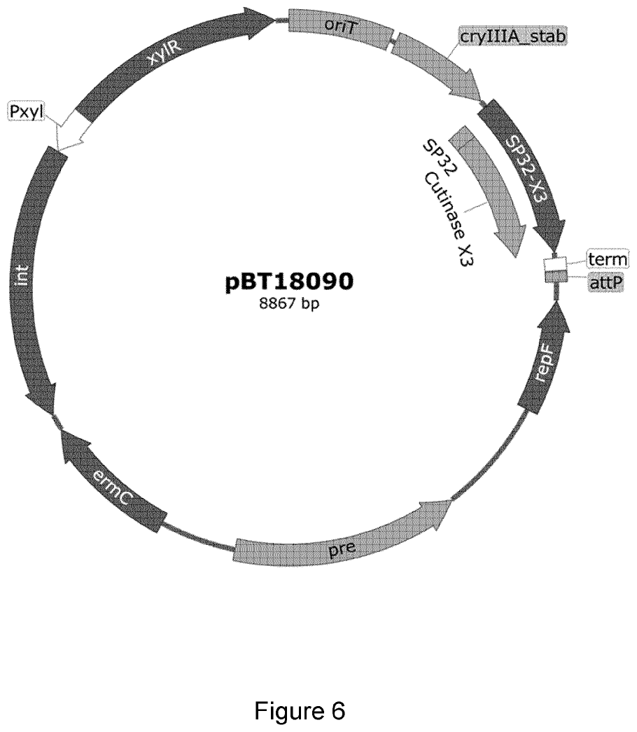

Plasmid pBT18089 and pBT18090 was constructed for insertion of a gene encoding cutinase variants X2 and X3 the signal peptide of Bacillus pumilus putative DUF3298 (designated by name SP32-X2 and SP32-X3, respectively) into the genome of a Bacillus host using the site-specific recombinase-mediated method described above. Construction of expression cassettes for SP32-X2 and SP32-X3 has been carried out according to the construction of SP32-X1 described in Example 2. The maps of pBT18089 and pBT18090 are shown in FIG. 5 and FIG. 6 respectively. Plasmid pBT18089 and pBT18090 was introduced into conjugation donor strain Bacillus subtilis pp 3724 by transformation, resulting in strain BT18089 and BT18090. Using conjugation donor strain BT18089 and BT18090, plasmid pBT18089 or pBT18090 was introduced by conjugation into a derivative of Bacillus licheniformis AN1302 comprising two chromosomal target sites for insertion of the plasmid as described above. Integration of SP32-X2 or SP32-X3 gene was confirmed by PCR analysis. One B. licheniformis integrant with the SP32-X2 or SP32-X3 gene inserted at two chromosomal loci was designated BT18093 and BT18095, respectively.

### Example 7. Comparison of Cutinase Production by Bacillus licheniformis Integrants Expressing Cutinase Variants Either with the Amyl Signal Peptide or with the SP32 Signal Peptide

B. licheniformis strains AN2783, BT18014, BT18093 and BT18095 were cultivated in BioLector®, and cutinase production by the strains was compared using activity assay. Relative total cutinase product is shown in Table 3. Using the SP32 signal peptide, cutinase yield was increased 2.5-fold and 2.2-fold in comparison to using the amyL signal peptide for expression of cutinase X2 and cutinase X3, respectively. In conclusion, SP32 increased the yield for cutinase X1, and also for its variants, namely cutinase X2 and cutinase X3.

TABLE 3
Relative cutinase product for B. licheniformis strains
expressing cutinase variants X2 and X3 (N = 6).

| | | Number of | Signal | Relative |
|---|---|---|---|---|
| | Cutinase | cutinase | Peptide | product |
| Strain | variants | gene copies | Source | yield |
| AN2783 | X2 | 2 | amyL | 0.9 ± 0.1 |
| BT18093 | X2 | 2 | SP32 | 2.5 ± 0.2 |
| BT18014 | X3 | 2 | amyL | 1.0 ± 0.0 |
| BT18095 | X3 | 2 | SP32 | 2.2 ± 0.2 |

### Example 8. Enzyme Assay Screening of Bacillus licheniformis SP Clones

The purpose of this experiment was to screen the Bacillus licheniformis SP clones using enzyme assay. The B. subtilis strains constructed in Example 8 were tested with respect to cutinase productivity in BioLector® batch cultivations. Cultivations were performed in a starch based slow carbon release medium, at a shaking frequency of 1000 rpm, 37° C. and 85% humidity in 48-well Flowerplates® (M2p-labs), covered with a Sealing Foil with Reduced Evaporation (M2p-Labs). After 72h, supernatants were harvested for subsequent cutinase activity measurements.

A library of 85 B. licheniformis clones was generated, which resulted in strains SP1 to SP85, comprising diverse SP sequences for the expression of cutinase X1. Each strain comprising one copy of the SP-cutinase fusion construct in its genome. The control strains with SPamyL are SP35, SP40, SP41 and SP65. The signal peptide SP32 with amino acid sequence of SEQ ID NO:2 is represented by strain SP32.

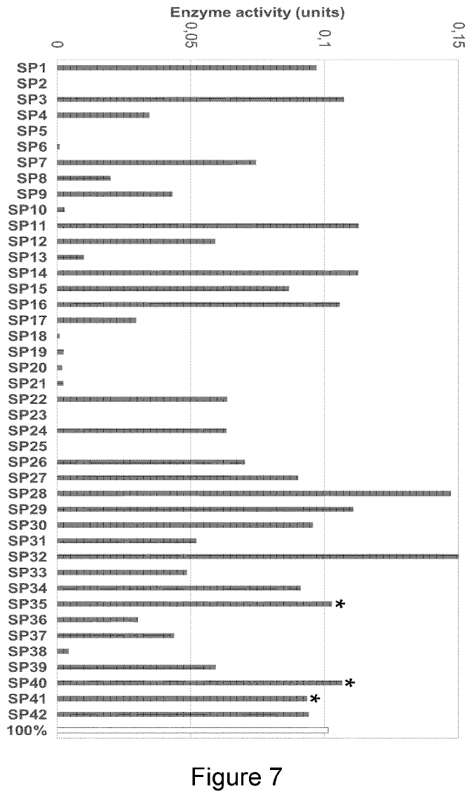

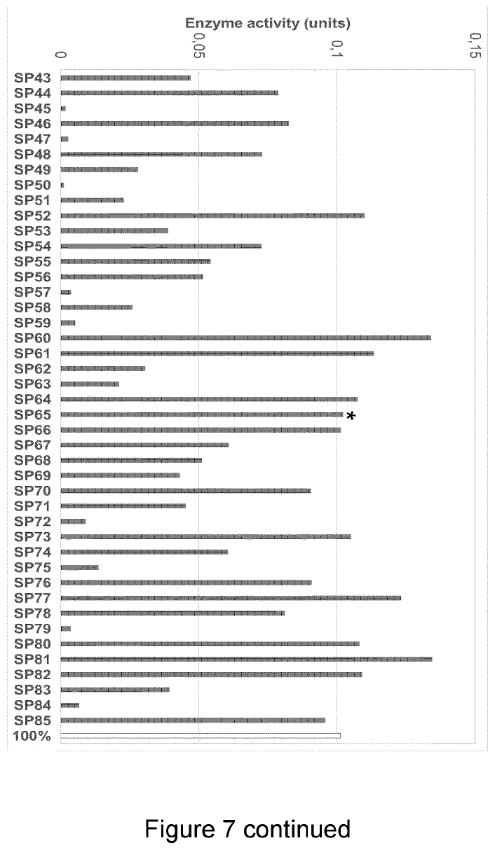

FIG. 7 and Table 4 show enzyme activity screening assay of tested Bacillus licheniformis SP clones 1-85.

In FIG. 7 Bacillus licheniformis clones with SPamyl are marked with “*” (=SP35, SP40, SP41, and SP65). Grey bars indicate Bacillus licheniformis SP clones 1-85. White bar indicates average cutinase activity of Bacillus licheniformis clones with SPamyL.

As can be seen in FIG. 7 and Table 4, most of the screened SP sequences result in decreased cutinase yield relative to the SPamyL. Only few SP sequences show increased cutinase yield, including SP32 which shows ca. 1.5-fold increased cutinase X1 expression compared to SPamyL. Also, amongst all screened signal peptides, SP32 showed the highest cutinase X1 expression. The second highest cutinase X1 expression is found for clone SP28 (SP28 has the same SP sequence as SP14 and SP60 as confirmed by DNA sequencing).

Both clones SP28 and SP32, together with control clone SP35 (SPamyL), were selected for fed-batch cultivation for further studies (Example 9).

TABLE 4
Relative cutinase activity for clones with SP1 to SP85.

| SP/clone number | relative enzyme activity |
|---|---|
| SP1 | 0.097033 |
| SP2 | −0.001144 |
| SP3 | 0.10721 |
| SP4 | 0.034421 |
| SP5 | −0.004629 |
| SP6 | 0.001087 |
| SP7 | 0.074291 |
| SP8 | 0.02 |
| SP9 | 0.043165 |
| SP10 | 0.002913 |
| SP11 | 0.112696 |
| SP12 | 0.058979 |
| SP13 | 0.01 |
| SP14 | 0.112664 |
| SP15 | 0.086522 |
| SP16 | 0.105658 |
| SP17 | 0.029588 |
| SP18 | 0.001006 |
| SP19 | 0.002448 |
| SP20 | 0.001947 |
| SP21 | 0.00229 |
| SP22 | 0.06356 |
| SP23 | 0 |
| SP24 | 0.063288 |
| SP25 | 2.96E−05 |
| SP26 | 0.070186 |
| SP27 | 0.090097 |
| SP28 | 0.147139 |
| SP29 | 0.110689 |
| SP30 | 0.095617 |
| SP31 | 0.052001 |
| SP32 | 0.151618 |
| SP33 | 0.048431 |
| SP34 | 0.091079 |
| SP35(SPamyL) | 0.102782 |
| SP36 | 0.030288 |
| SP37 | 0.043652 |
| SP38 | 0.004419 |
| SP39 | 0.059126 |
| SP40(SPamyL) | 0.106446 |
| SP41(SPamyL) | 0.09355 |
| SP42 | 0.094063 |
| SP43 | 0.047123 |
| SP44 | 0.078634 |
| SP45 | 0.001796 |
| SP46 | 0.082572 |
| SP47 | 0.0027 |
| SP48 | 0.072804 |
| SP49 | 0.027954 |
| SP50 | 0.00129 |
| SP51 | 0.022936 |
| SP52 | 0.110093 |
| SP53 | 0.038891 |
| SP54 | 0.072653 |
| SP55 | 0.054277 |
| SP56 | 0.051602 |
| SP57 | 0.003764 |
| SP58 | 0.026001 |
| SP59 | 0.005443 |
| SP60 | 0.134099 |
| SP61 | 0.113375 |
| SP62 | 0.030703 |
| SP63 | 0.021261 |
| SP64 | 0.107543 |
| SP65(SPamyL) | 0.10233 |
| SP66 | 0.101363 |
| SP67 | 0.060818 |
| SP68 | 0.051061 |
| SP69 | 0.043136 |
| SP70 | 0.090606 |
| SP71 | 0.045295 |
| SP72 | 0.009154 |
| SP73 | 0.105067 |
| SP74 | 0.060539 |
| SP75 | 0.013809 |
| SP76 | 0.090854 |
| SP77 | 0.123257 |
| SP78 | 0.081135 |
| SP79 | 0.003667 |
| SP80 | 0.108117 |
| SP81 | 0.13442 |
| SP82 | 0.109206 |
| SP83 | 0.039438 |
| SP84 | 0.006751 |
| SP85 | 0.095831 |
| 100% (=average of enzyme | 0.101277 |
| activities of SPamyL clones) | |

### Example 9: Fed-Batch Cultivation with Increased Cutinase Yields for SP 32 Standard Fed-Batch Cultivation Procedure

All growth media were sterilized by methods known in the art. Unless otherwise described, tap water was used. The ingredient concentrations referred to in the below recipes are before any inoculation.

First inoculum medium: SSB4 agar. Soy peptone SE50MK (DMV) 10 g/l; sucrose 10 g/l;
di-sodiumhydrogenphosphate, 2H₂O 5 g/l; potassiumdihydrogenphosphate 2 g/l; citric acid 0.2 g/l; vitamins (Thiamin-hydrochlorid 1 1.4 mg/l; riboflavin 0.95 mg/l; nicotinic amide 7.8 mg/l; calcium D-pantothenate 9.5 mg/l; pyridoxal-HCl 1.9 mg/l; D-biotin 0.38 mg/l; folic acid 2.9 mg/l); trace metals (MnSO₄, H₂O 9.8 mg/l; FeSO₄, 7H₂O 39.3 mg/l; CuSO₄, 5H₂O 3.9 mg/l; ZnSO₄, 7H₂O 8.2 mg/l); Agar 25 g/l. Use of deionized water. pH adjusted to pH 7.3 to 7.4 with NaOH.

Transfer buffer. M-9 buffer (deionized water is used): Di-sodiumhydrogenphosphate, 2H20 8.8 g/l; potassiumdihydrogenphosphate 3 g/l; sodium chloride 4 g/l; magnesium sulphate, 7H20 0.2 g/l.

Inoculum shake flask medium (concentration is before inoculation): PRK-50:10 g/l soy grits; di-sodiumhydrogenphosphate, 2H2O 5 g/l; pH adjusted to 8.0 with NaOH/H₃PO4 before sterilization.

Make-up medium (concentration is before inoculation): Tryptone (casein hydrolysate from Difco) 30 g/l; magnesium sulphate, 7H₂O 4 g/l; di-potassiumhydrogenphosphate 7 g/l; di-sodiumhydrogenphosphate, 2H₂O 7 g/l; di-ammoniumsulphate 4 g/l; potassiumsulphate 5 g/l; citric acid 0.78 g/l; vitamins (Thiamin-hydrochloride 34.2 mg/l; Riboflavin 2.8 mg/l; Nicotinic amide 23.3 mg/l; calcium D-pantothenate 28.4 mg/l; pyridoxal-HCl 5.7 mg/l; D-biotin 1.1 mg/l; folic acid 2.5 mg/l); trace metals (MnSO₄, H₂O 39.2 mg/l; FeSO₄, 7H₂O 157 mg/l; CuS04, 5H₂O 15.6 mg/l; ZnSO₄, 7H₂O 32.8 mg/l); Antifoam (SB2121) 1.25 ml/l; pH adjusted to 6.0 with NaOH/H₃P04 before sterilization.
Feed medium: Sucrose 708 g/l;
Inoculum steps: First the strain was grown on SSB-4 agar slants 1 day at 37° C. The agar was then washed with M-9 buffer, and the optical density (OD) at 650 nm of the resulting cell suspension was measured. The inoculum shake flask (PRK-50) was inoculated with an inoculum of OD (650 nm)×ml cell suspension=0.1. The shake flask was incubated at 37° C. at 300 rpm for 20 hr. The fermentation in the main fermentor (fermentation tank) was started by inoculating the main fermentor with the growing culture from the shake flask. The inoculated volume was 11% of the make-up medium (80 ml for 720 ml make-up media).

Standard lab fermentors were used equipped with a temperature control system, pH control with ammonia water and phosphoric acid, dissolved oxygen electrode to measure oxygen saturation through the entire fermentation.

Fermentation parameters: Temperature: 30-42° C.; The pH was kept between 6.8 and 7.2 using ammonia water and phosphoric acid; Control: 6.8 (ammonia water); 7.2 phosphoric acid;

Aeration: 1.5 liter/min/kg broth weight.

Agitation: 1500 rpm.

Feed strategy: 0 hr. 0.05 g/min/kg initial broth after inoculation; 8 hr. 0.156 g/min/kg initial broth after inoculation; End 0.156 g/min/kg initial broth after inoculation.

Experimental setup: The cultivation was run for five days with constant agitation, and the oxygen tension was followed on-line in this period. The different strains were compared side by side. Measurements of cutinase activities was performed by the method described above.

### Cutinase Yields

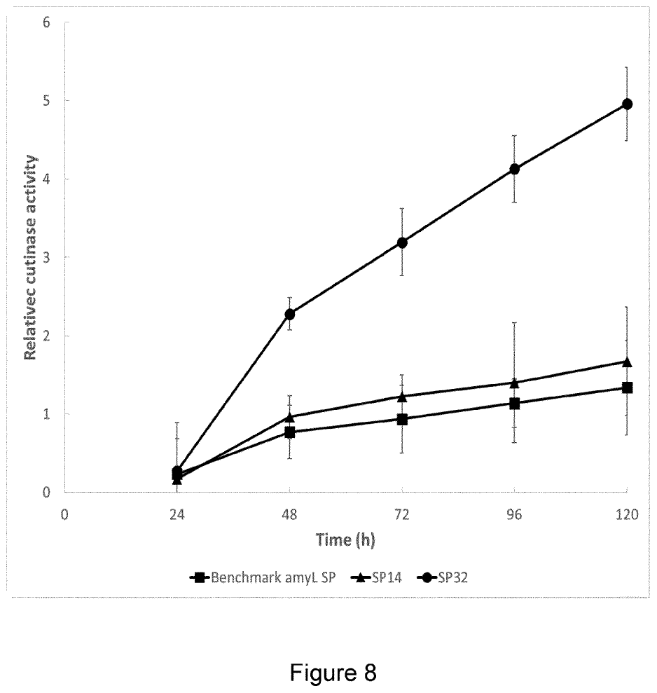

Relative cutinase yield for clones SP28, SP32, and SP35 (SPamyL control) during the course of 120h of fermentation is shown in FIG. 8. All three clones showed an increase in cutinase yield over time. However, clone SP32 having SEQ ID NO:2 as SP sequence fused to the cutinase X1 gene showed a significant increase of cutinase yield relative to both SP28 and SP35. After 120 hours of fermentation, the cutinase yield of clone SP32 was 4- to 5-fold increased compared to cutinase yield of SP28 and SP35. In comparison, only a 15-20% cutinase yield increase was observed for SP28 when compared to SPamyL.

Surprisingly, already 48 hours after fermentation the cutinase yield of clone SP32 has doubled when compared to SP28 and SPamyL. The difference in cutinase yield between SP32 and SP28 was particularly surprising since both clones showed a similar increase in cutinase yield during the small-scale screening assay (FIG. 7). Thus, the superior performance of SP32 with the signal peptide sequence of SEQ ID NO:2 for the expression of cutinases as shown in FIG. 8 was totally unexpected.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### LIST OF EMBODIMENTS

The invention is further defined by the following numbered embodiments:

[1] A nucleic acid construct comprising:
a) a first polynucleotide encoding a signal peptide from a bacterial DUF3298 domain-containing polypeptide; and
b) a second polynucleotide encoding a polypeptide having cutinase activity;
wherein the first polynucleotide and the second polynucleotide are operably linked in translational fusion.

[2] The nucleic acid construct according to embodiment 1, wherein the nucleic acid construct further comprises a heterologous promoter, and wherein said promoter, the first polynucleotide, and the second polynucleotide are operably linked.

[3] The nucleic acid construct according to embodiment 2, wherein the promoter is a P3 promoter or a P3-based promoter, preferably the heterologous promoter is a tandem promoter comprising the P3 promoter or is a tandem promoter derived from the P3 promoter.

[4] The nucleic acid construct according to any of embodiments 2 to 3, wherein the promoter is operably linked to an mRNA stabilizer region; preferably the mRNA stabilizer region is the cryIIIA mRNA stabilizer region, more preferably the cryIIIA mRNA stabilizer region is the cryIIIA mRNA stabilizer region of SEQ ID NO:15.

[5] The nucleic acid construct according to any of the preceding embodiments, wherein the signal peptide is a naturally occurring signal peptide, or a functional fragment or functional variant of a naturally occurring signal peptide.

[6] The nucleic acid construct according to any of the preceding embodiments, wherein the signal peptide is obtained from a DUF3298 domain-containing polypeptide expressed by a Gram-positive bacterium, preferably by a Bacillus species.

[7] The nucleic acid construct according to any of the preceding embodiments, wherein the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO: 1; most preferably the polynucleotide comprises, consists essentially of, or consists of the mature polypeptide coding sequence of SEQ ID NO: 1.

[8] The nucleic acid construct according to any of the preceding embodiments, wherein the signal peptide is obtained from a DUF3298 domain-containing polypeptide expressed by a Bacillus species selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

[9] The nucleic acid construct according to any of the preceding embodiments, wherein the signal peptide is obtained from a DUF3298 domain-containing polypeptide expressed by Bacillus licheniformis, Bacillus subtilis or Bacillus pumilus.

[10] The nucleic acid construct according to any of the preceding embodiments, wherein the signal peptide is obtained from a DUF3298 domain-containing polypeptide expressed by Bacillus pumilus.

[11] The nucleic acid construct according to any of the preceding embodiments, wherein the signal peptide is obtained from a bacterial DUF3298 domain-containing polypeptide having a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 4.

[12] The nucleic acid construct according to any of the preceding embodiments, wherein the bacterial DUF3298 domain-containing polypeptide comprises, consists essentially of, or consists of SEQ ID NO: 4.

[13] The nucleic acid construct according to any of the preceding embodiments, wherein the signal peptide has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO:2.

[14] The nucleic acid construct according to any of the preceding embodiments, wherein the signal peptide comprises, consists essentially of, or consists of SEQ ID NO:2.

[15] The nucleic acid construct according to any of the preceding embodiments, wherein the N- and/or C-terminal end of the signal peptide has been extended by addition of one or more amino acids.

[16] The nucleic acid construct according to any of embodiments 1 to 14, wherein the signal peptide is a fragment of the signal peptides of any of embodiments 1 to 14.

[17] The nucleic acid construct according to any of the preceding embodiments, wherein polynucleotide encoding the polypeptide having cutinase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:5; most preferably the polynucleotide comprises, consists essentially of, or consists of the mature polypeptide coding sequence of SEQ ID NO:5.

[18] The nucleic acid construct according to any of the preceding embodiments, wherein the polypeptide having cutinase activity is a microbial polypeptide.

[19] The nucleic acid construct according to any of the preceding embodiments, wherein the polypeptide having cutinase activity is a bacterial polypeptide.

[20] The nucleic acid construct according to any one of embodiments 18 to 19, wherein the polypeptide having cutinase activity is obtained from Thermobifida cellulosilytica DSM44535.

[21] The nucleic acid construct according to any one of embodiments 18 to 19, wherein the polypeptide having cutinase activity is obtained from Thermobifida halotolerans.

[22] The nucleic acid construct according to any one of embodiments 18 to 19, wherein the polypeptide having cutinase activity is obtained from Thermobifida fusca.

[23] The nucleic acid construct according to any one of embodiments 18 to 19, wherein the polypeptide having cutinase activity is obtained from Thermobifida alba.

[24] The nucleic acid construct according to any one of embodiments 18 to 19, wherein the polypeptide having cutinase activity is derived from Fusarium solani.

[25] The nucleic acid construct according to any one of embodiments 18 to 19, wherein the polypeptide having cutinase activity is derived from or Fusarium solani pisi.

[26] The nucleic acid construct according to any one of embodiments 18 to 19, wherein the polypeptide having cutinase activity is obtained from Bacillus subtilis.

[27] The nucleic acid construct according to any one of embodiments 18 to 19, wherein the polypeptide having cutinase activity is obtained from Thielavia terrestris.

[28] The nucleic acid construct according to any one of embodiments 18 to 19, wherein the polypeptide having cutinase activity is obtained from Humicola insolens.

[29] The nucleic acid construct according to any one of embodiments 18 to 19, wherein the polypeptide having cutinase activity is obtained from Glomerella cingulate.

[30] The nucleic acid construct according to any preceding embodiments, wherein the polypeptide having cutinase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide of SEQ ID NO:6.

[31] The nucleic acid construct according to embodiment 30, wherein the polypeptide having cutinase activity comprises, consists essentially of, or consists of the mature polypeptide of SEQ ID NO:6.

[32] The nucleic acid construct according to any of the preceding embodiments, wherein the N- and/or C-terminal end of the cutinase has been extended by addition of one or more amino acids.

[33] The nucleic acid construct according to any of embodiments 1 to 31, wherein the cutinase is a fragment of the signal peptides of any of embodiments 1 to 31.

[34] An expression vector comprising a nucleic acid construct according to any of embodiments 1 to 33.

[35] A bacterial host cell comprising in its genome:
a) a nucleic acid construct according to any of embodiments 1 to 33; and/or
b) an expression vector according to embodiment 34.

[36] The bacterial host cell of embodiment 35, wherein the bacterial host cell is a Gram-positive host cell.

[37] The bacterial host cell of any of embodiments 35 to 36, wherein the bacterial host cell is a Bacillus cell.

[38] The bacterial host cell of any of embodiments 35 to 37, wherein the bacterial host cell is a Bacillus cell selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cell.

[39] The bacterial host cell of any of embodiments 35 to 38, wherein the bacterial host cell is a Bacillus licheniformis cell.

[40] The bacterial host cell of any of embodiments 35 to 39, wherein the host cell comprises one copy of the nucleic acid construct and/or the expression vector, or wherein the host cell comprises at least two copies of the nucleic acid construct and/or the expression vector, such as two copies, three copies, four copies or more than four copies.

[41] The bacterial host cell of any of embodiments 35 to 40, wherein the host cell has increased cutinase yield relative to a control host cell lacking the signal peptide, being otherwise isogenic, when cultivated under identical conditions.

[42] The bacterial host cell of any of embodiments 35 to 41, wherein the control host cell encodes a amyL signal peptide fused to the cutinase.

[43] The bacterial host cell of any of embodiments 35 to 42, wherein the amyL signal peptide fused to the cutinase has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to SEQ ID NO:8; most preferably the amyL signal peptide comprises, consists essentially of, or consists of SEQ ID NO:8.

[44] The bacterial host cell of any of embodiments 35 to 43, wherein the increased cutinase yield of the host cell is at least 1.5-fold, 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, at least 5-fold, least 5.1-fold, at least 5.2-fold, at least 5.3-fold, at least 5.4-fold, or at least 5.5-fold relative to the control host cell.

[45] The bacterial host cell of any of embodiments 35 to 44, wherein the cutinase yield is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, at least 400%, at least 405%, at least 410%, at least 415%, at least 420%, at least 425%, at least 430%, at least 435%, at least 440%, at least 445%, at least 450%, at least 455%, at least 460%, at least 465%, at least 470%, at least 475%, at least 480%, at least 485%, at least 490%, at least 495%, or at least 500%, relative to the cutinase yield in the control host cell which does not comprise the SP32 signal peptide, when cultivated under identical conditions. Preferably, the control host cell is otherwise isogenic to the host cell.

[46] The bacterial host cell of any of embodiments 35 to 45, wherein the increased cutinase yield is measured or achieved after a cultivation time of at least 24 hours, at least 48 hours, at least 72 hours, at least 96 hours, or at least 120 hours.

[47] The bacterial host cell of any of embodiments 35 to 46, wherein the increased cutinase yield is measured or achieved after cultivation in batch-mode, fed-batch mode, or continuous mode, preferably fed-batch mode.

[48] A method of producing a polypeptide having cutinase activity, the method comprising:
a) cultivating a bacterial host cell according to any of embodiments 35 to 47 under conditions conducive for production of the polypeptide having cutinase activity; and optionally
b) recovering the polypeptide having cutinase activity.

[49] A method of producing a whole broth formulation or cell culture composition comprising a polypeptide having cutinase activity, the method comprising:
a) cultivating a bacterial host cell according to any of embodiments 35 to 47 under conditions conducive for production of the polypeptide having cutinase activity; and
b) filtering the cultivation broth to recover the polypeptide having cutinase activity.

[50] The method of any of embodiments 48-49, wherein the cultivation is a fed-batch process.

[51] The method of any one of embodiments 48-50, wherein the cultivation is carried out over a period of at least 48 hours, at least 72 hours, at least 96 hours, or at least 120 hours.

[52] Use of a filtered broth in a PET degradation process, wherein the broth is obtained by the method according to any of embodiments 48 to 51.

[53] Use of a fermentation broth in a PET degradation processes, the broth comprising a polypeptide having cutinase activity and a host cell according to any of embodiments 35 to 47.

[54] Use according to embodiment 53, wherein the fermentation broth is used directly in a PET degradation process, without further filtration or purification of the polypeptide having cutinase activity.

[55] Use according to any one of embodiments 52 to 54, wherein the host cells are inactivated, preferably the host cells have a low cell viability, more preferably the host cells are killed.

1-15. (canceled)

16. A nucleic acid construct comprising:
a) a first polynucleotide encoding a signal peptide having a sequence identity of at least 60% to SEQ ID NO:2; and
b) a second polynucleotide encoding a polypeptide having cutinase activity;
wherein the first polynucleotide and the second polynucleotide are operably linked in translational fusion.

17. The nucleic acid construct according to claim 16, wherein the nucleic acid construct further comprises a heterologous promoter, and wherein said promoter, the first polynucleotide, and the second polynucleotide are operably linked.

18. The nucleic acid construct according to claim 17, wherein the promoter is a P3 promoter or a P3-based promoter.

19. The nucleic acid construct according to claim 16, wherein the signal peptide is a naturally occurring signal peptide, or a functional fragment or functional variant of a naturally occurring signal peptide.

20. The nucleic acid construct according to claim 16, wherein the signal peptide is from a DUF3298 domain-containing polypeptide expressed by a Bacillus species.

21. 6. The nucleic acid construct according to claim 16, wherein the signal peptide is has a sequence identity of at least 80% to SEQ ID NO: 4.

22. The nucleic acid construct according to claim 16, wherein the signal peptide comprises or consists of SEQ ID NO: 4.

23. The nucleic acid construct according to claim 16, wherein the signal peptide has a sequence identity of at least 65% to SEQ ID NO: 2.

24. The nucleic acid construct according to claim 16, wherein the signal peptide comprises or consists of SEQ ID NO: 2.

25. The nucleic acid construct according to claim 16, wherein the polypeptide having cutinase activity is a microbial polypeptide.

26. The nucleic acid construct according to claim 16, wherein the polypeptide having cutinase activity a bacterial polypeptide.

27. The nucleic acid construct according to claim 16, wherein the polypeptide having cutinase activity is obtained from Thermobifida cellulosilytica, Thermobifida halotolerans, Thermobifida fusca, Thermobifida alba, Fusarium solani, Fusarium solani pisi, Bacillus subtilis, Humicola insolens, Glomerella cingulate, or Thielavia terrestris.

28. The nucleic acid construct according to claim 16, wherein the polypeptide having cutinase activity has a sequence identity of at least 80% to the mature polypeptide of SEQ ID NO: 6.

29. The nucleic acid construct according to claim 16, wherein the polypeptide having cutinase activity comprises or consists of the mature polypeptide of SEQ ID NO: 6.

30. An expression vector comprising a nucleic acid construct according to claim 16.

31. A bacterial host cell comprising in its genome the expression vector according to claim 30.

32. The bacterial host cell of claim 31, wherein the bacterial host cell is a Gram-positive host cell.

33. A method of producing a polypeptide having cutinase activity, the method comprising cultivating a bacterial host cell according to claim 31 under conditions conducive for production of the polypeptide having cutinase activity.

34. The method of claim 33, further comprising recovering the polypeptide having cutinase activity.


SEQUENCE LISTING (17 sequences)

SEQ ID NO: 1 - DNA, 84 bp, Bacillus pumilus
  source 1..84: mol_type = genomic DNA
atgacaatgaaaaaaggcctaatgatcagcctgctcattttttcgctgattgtttcgttgtttgcgccagttggatctgcggat

SEQ ID NO: 2 - protein, 28 aa, Bacillus pumilus
MTMKKGLMISLLIFSLIVSLFAPVGSAD

SEQ ID NO: 3 - DNA, 678 bp, Bacillus pumilus
  source 1..678: mol_type = genomic DNA
atgacaatgaaaaaaggcctaatgatcagcctgctcattttttcgctgattgtttcgttgtttgcgccagttggatctgcggatgccaaacaagcaaaaaaagcaaaagtaagcacacactattataaaaagaaaaaaacgttcaaatatgcacaggtgaaaaacctaagcaaggcgcatatgaaaaccatcaatcgtgatttgaaagaagatatatccttctcctattctacgtacttgttcttaaaggacgaagcgaaaaaggaaggctttgggaagatcacgtacgaaagatcgtttaaaacgaaatttaacaatgggaagaagctcagtatcttaaactatgactacatgaatgatggtggaatgcatggggatacgatggtttccagcatgaactatgtgttaacaaagaaaaagacaaagcagctgttcttgacagacgttgtgaaaacaaagaaaaaacgtacgaaagtaagagattatatttataactacacaaagaaacataaaagcaaattctatgcgaacctgaaaaaaagtgacattgaaatcaactacaacacgcagtattacttcacgaaaaagggaattgctgttgtcttccagccatatgaaatttcaccttattatgtaggaattcctatgattcaagttcctgcgaaggtatataaataa

SEQ ID NO: 4 - protein, 225 aa, Bacillus pumilus
MTMKKGLMISLLIFSLIVSLFAPVGSADAKQAKKAKVSTHYYKKKKTFKYAQVKNLSKAHMKTINRDLKEDISFSYSTYLFLKDEAKKEGFGKITYERSFKTKFNNGKKLSILNYDYMNDGGMHGDTMVSSMNYVLTKKKTKQLFLTDVVKTKKKRTKVRDYIYNYTKKHKSKFYANLKKSDIEINYNTQYYFTKKGIAVVFQPYEISPYYVGIPMIQVPAKVYK

SEQ ID NO: 5 - DNA, 777 bp, synthetic construct
  source 1..777: mol_type = other DNA
tcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtag

SEQ ID NO: 6 - protein, 258 aa, synthetic construct
  REGION 1..258: note = cutinase X1
SNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHSMGGGGTLRIAEQNPSLKAAVPLTPWHTDKTFNTSVPVLIIGAEADTVAPVSQHAIPFYQNLPSTTPKVYVELCNASHIAPNSNNAAISVYTISWMKLWVDNDTRYRQFLCNVNDPALCDFRTNNRHCQ

SEQ ID NO: 7 - DNA, 84 bp, Bacillus licheniformis
  source 1..84: mol_type = genomic DNA
atgaaacaacaaaaacgactttacgcccgattgctcacgctgttatttgcgctgatcttcttgctgcctcattcggcagcagcg

SEQ ID NO: 8 - protein, 28 aa, Bacillus licheniformis
MKQQKRLYARLLTLLFALIFLLPHSAAA

SEQ ID NO: 9 - DNA, 864 bp, synthetic construct
  misc_feature 1..864: note = SP32-X1 coding sequence
  source 1..864: mol_type = other DNA
atgacaatgaaaaaaggcctaatgatcagcctgctcattttttcgctgattgtttcgttgtttgcgccagttggatctgcggatgcgtcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtag

SEQ ID NO: 10 - protein, 287 aa, synthetic construct
  REGION 1..287: note = SP32-X1
MTMKKGLMISLLIFSLIVSLFAPVGSADASNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHSMGGGGTLRIAEQNPSLKAAVPLTPWHTDKTFNTSVPVLIIGAEADTVAPVSQHAIPFYQNLPSTTPKVYVELCNASHIAPNSNNAAISVYTISWMKLWVDNDTRYRQFLCNVNDPALCDFRTNNRHCQ

SEQ ID NO: 11 - DNA, 861 bp, synthetic construct
  misc_feature 1..861: note = SPamyL-X1 coding sequence
  source 1..861: mol_type = other DNA
atgaaacaacaaaaacgactttacgcccgattgctcacgctgttatttgcgctgatcttcttgctgcctcattcggcagcagcgtcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtag

SEQ ID NO: 12 - protein, 314 aa, synthetic construct
  REGION 1..314: note = SPamyL-X1
MKQQKRLYARLLTLLFALIFLLPHSAAAMKQQKRLYARLLTLLFALIFLLPHSAAASNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHSMGGGGTLRIAEQNPSLKAAVPLTPWHTDKTFNTSVPVLIIGAEADTVAPVSQHAIPFYQNLPSTTPKVYVELCNASHIAPNSNNAAISVYTISWMKLWVDNDTRYRQFLCNVNDPALCDFRTNNRHCQ

SEQ ID NO: 13 - DNA, 87 bp, synthetic construct
  misc_feature 1..87: note = SP32
  source 1..87: mol_type = other DNA
atgacaatgaaaaaaggcctaatgatcagcctgctcattttttcgctgattgtttcgttgtttgcgccagttggatctgcggatgcg

SEQ ID NO: 14 - protein, 29 aa, synthetic construct
  REGION 1..29: note = SP32
MTMKKGLMISLLIFSLIVSLFAPVGSADA

SEQ ID NO: 15 - DNA, 557 bp, synthetic construct
  misc_feature 1..557: note = cryIIIA stabilizer
  source 1..557: mol_type = other DNA
taatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatg

SEQ ID NO: 16 - DNA, 2120 bp, synthetic construct
  misc_feature 1..2120: note = cassette SP32-X1
  source 1..2120: mol_type = other DNA
gcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatggagctcagaggaggagagtgagtaatgacaatgaaaaaaggcctaatgatcagcctgctcattttttcgctgattgtttcgttgtttgcgccagttggatctgcggatgcgtcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtagtagccgcgtgctagcggccgcgtcgactagaagagcagagaggacggatttcctgaaggaaatccgtttttttattttgcccgtcttataaatttcgttgtccaactcgcttaattgcgagtttttatttcgtttatttcaattaaggtaactaaagatcctctagagtcgattatgtcttttgcgcagtcggcttaaaccagttttcgctggtgcgaaaaaagagtgtcttgtgacacctaaattcaaaatcta

SEQ ID NO: 17 - DNA, 2145 bp, synthetic construct
  misc_feature 1..2145: note = cassette SPamyl-X1
  source 1..2145: mol_type = other DNA
gcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatagaaaggaggtgatccagccgcaccttatgaaaaatcattttatcagtttgaaaattatgtattatgtggatcctacgcgttcatatgtatcacattgaaaggggaggagaatcatgaaacaacaaaaacgactttacgcccgattgctcacgctgttatttgcgctgatcttcttgctgcctcattcggcagcagcggcatcaaacccgtatcaaagaggaccgaatcccacaagatccgccttaacagcggacggaccattttctgtcgcgacctatactgtgagtcgtctttctgtgagcggatttggcggtggcgttatctactatccgaccgggacgagcctgacttttggagggattgcgatgtctccaggatacactgccgacgcctcttctttggcctggttaggaagacgcttggcttcccatgggttcgtggttttggtgatcaatacaaacagtagatttgatggaccggattcgcgtgcctcgcaactgtcagctgccctcaactacctcagaacttcctctccctccgctgtaagagcacgcttggatgccaacagattggccgtagcaggacattcaatgggaggaggcggtacgctccggattgcggaacaaaatccgtctttgaaagccgcagtaccgttgacgccgtggcacacagacaaaacctttaacacctccgttcccgtgttgatcattggagccgaagccgatacagttgcaccggtcagccaacatgctattccgttttaccagaacttaccgtctacaacaccgaaagtctacgtcgagctctgtaatgcatcccatattgcaccgaattcgaacaatgccgcaattagcgtgtacactatttcatggatgaaactctgggtggacaatgatacaagatatcggcagtttttgtgcaacgtaaatgaccctgcgctttgcgactttcggacaaacaaccgccattgccagtagtagccgcgtgctagcggccgcgtcgactagaagagcagagaggacggatttcctgaaggaaatccgtttttttattttgcccgtcttataaatttcgttgtccaactcgcttaattgcgagtttttatttcgtttatttcaattaaggtaactaaagatcctctagagtcgattatgtcttttgcgcagtcggcttaaaccagttttcgctggtgcgaaaaaagagtgtcttgtgacactcttaaattcaaaatcta